(19) United States
(12) Patent Application Publication
(10) Pub. No.: US 2025/0090135 A1
(43) Pub. Date: Mar. 20, 2025

(54) DIAGNOSIS SUPPORT DEVICE, ULTRASOUND ENDOSCOPE, DIAGNOSIS SUPPORT METHOD, AND PROGRAM

(71) Applicant: FUJIFILM CORPORATION, Minato-ku, Tokyo (JP)

(72) Inventor: Toshihiro USUDA, Kanagawa (JP)

(21) Appl. No.: 18/969,284

(22) Filed: Dec. 5, 2024

Related U.S. Application Data
(63) Continuation of application No. PCT/JP2023/020889, filed on Jun. 5, 2023.

(30) Foreign Application Priority Data
Jun. 29, 2022 (JP) 2022-105152

(51) Int. Cl. A61B 8/08; A61B 8/00

(52) U.S. Cl. CPC A61B 8/085; A61B 8/44; A61B 8/461; A61B 8/5215

(57) ABSTRACT

A diagnosis support device includes a processor. The processor acquires an ultrasound image, displays the acquired ultrasound image on a display device, and displays, in the ultrasound image, a first mark capable of specifying a lesion region detected from the ultrasound image within the ultrasound image and a second mark capable of specifying an organ region detected from the ultrasound image within the ultrasound image. The first mark is displayed in a state of being emphasized more than the second mark.

### CROSS-REFERENCE TO RELATED APPLICATIONS

This application is a continuation application of International Application No. PCT/JP2023/020889, filed Jun. 5, 2023, the disclosure of which is incorporated herein by reference in its entirety. Further, this application claims priority from Japanese Patent Application No. 2022-105152, filed Jun. 29, 2022, the disclosure of which is incorporated herein by reference in its entirety.

### BACKGROUND

### 1. Technical Field

The technology of the present disclosure relates to a diagnosis support device, an ultrasound endoscope, a diagnosis support method, and a program.

### 2. Related Art

JP2021-185970A discloses an image processing device that processes a medical image. The image processing device disclosed in JP2021-185970A comprises a detection unit that detects a lesion candidate region, a validity evaluation unit that evaluates validity of the lesion candidate region by using a normal tissue region corresponding to the detected lesion candidate region, and a display unit that determines a content to be displayed to a user by using an evaluation result.

JP2015-154918A discloses a lesion detection device. The lesion detection device disclosed in JP2015-154918A includes a lesion candidate detector that detects lesion candidates in a medical video, a peripheral object detector that detects an anatomical object in the medical video, a lesion candidate verifier that verifies the lesion candidates based on anatomical context information including relationship information between positions of the lesion candidates and a position of the anatomical object, and a candidate remover that removes a false positive lesion candidate from the detected lesion candidates based on a verification result of the lesion candidate verifier.

JP2021-180730A discloses an ultrasound diagnostic device. The ultrasound diagnostic device disclosed in JP2021-180730A includes a detection unit that detects lesion part candidates based on a frame data sequence obtained by transmitting and receiving ultrasound, and a notification unit that displays a mark for notifying of the lesion part candidates on an ultrasound image generated from the frame data sequence based on a detection result of the detection unit, the notification unit changing a display aspect of the mark in accordance with a degree of probability that the lesion part candidate is a lesion part. In addition, the notification unit includes a calculation unit that calculates a reliability degree indicating the degree of probability that the lesion part candidate is the lesion part based on the frame data sequence, and a control unit that changes the display aspect of the mark in accordance with the reliability degree. In addition, in a case in which the reliability degree is low, the control unit changes the display aspect such that the mark is less conspicuous than in a case in which the reliability degree is high.

### SUMMARY

One embodiment according to the technology of the present disclosure provides a diagnosis support device, an ultrasound endoscope, a diagnosis support method, and a program that can suppress overlooking of a lesion region in a diagnosis using an ultrasound image.

A first aspect according to the technology of the present disclosure relates to a diagnosis support device comprising: a processor, in which the processor acquires an ultrasound image, displays the acquired ultrasound image on a display device, and displays, in the ultrasound image, a first mark capable of specifying a lesion region detected from the ultrasound image within the ultrasound image and a second mark capable of specifying an organ region detected from the ultrasound image within the ultrasound image, and the first mark is displayed in a state of being emphasized more than the second mark.

A second aspect according to the technology of the present disclosure relates to the diagnosis support device according to the first aspect, in which the first mark is a mark capable of specifying an outer edge of a first range in which the lesion region is present.

A third aspect according to the technology of the present disclosure relates to the diagnosis support device according to the second aspect, in which the first range is defined by a first rectangular frame that surrounds the lesion region.

A fourth aspect according to the technology of the present disclosure relates to the diagnosis support device according to the third aspect, in which the first rectangular frame is a rectangular frame that circumscribes the lesion region.

A fifth aspect according to the technology of the present disclosure relates to the diagnosis support device according to the third or fourth aspect, in which the first mark is a mark in which at least a part of the first rectangular frame is formed in a visually specifiable manner.

A sixth aspect according to the technology of the present disclosure relates to the diagnosis support device according to any one of the third to fifth aspects, in which the first rectangular frame surrounds the lesion region in a rectangular shape as seen in front view, and the first mark is composed of a plurality of first images assigned to a plurality of corners including at least opposite corners of four corners of the first rectangular frame.

A seventh aspect according to the technology of the present disclosure relates to the diagnosis support device according to any one of the first to sixth aspects, in which the second mark is a mark capable of specifying an outer edge of a second range in which the organ region is present.

An eighth aspect according to the technology of the present disclosure relates to the diagnosis support device according to the seventh aspect, in which the second range is defined by a second rectangular frame that surrounds the organ region.

A ninth aspect according to the technology of the present disclosure relates to the diagnosis support device according to the eighth aspect, in which the second rectangular frame is a rectangular frame that circumscribes the organ region.

A tenth aspect according to the technology of the present disclosure relates to the diagnosis support device according to the eighth or ninth aspect, in which the second mark is a mark in which at least a part of the second rectangular frame is formed in a visually specifiable manner.

An eleventh aspect according to the technology of the present disclosure relates to the diagnosis support device according to any one of the eighth to tenth aspects, in which the second rectangular frame surrounds the organ region in a rectangular shape as seen in front view, and the second mark is composed of a plurality of second images assigned to center portions of a plurality of sides including at least opposite sides of four sides of the second rectangular frame.

A twelfth aspect according to the technology of the present disclosure relates to the diagnosis support device according to any one of the first to eleventh aspects, in which the ultrasound image is a moving image including a plurality of frames, and in a case in which N is a natural number equal to or larger than 2, the processor displays the first mark in the ultrasound image in a case in which the lesion region is detected from N consecutive frames among the plurality of frames.

A thirteenth aspect according to the technology of the present disclosure relates to the diagnosis support device according to any one of the first to twelfth aspects, in which the ultrasound image is a moving image including a plurality of frames, and in a case in which M is a natural number equal to or larger than 2, the processor displays the second mark in the ultrasound image in a case in which the organ region is detected from M consecutive frames among the plurality of frames.

A fourteenth aspect according to the technology of the present disclosure relates to the diagnosis support device according to any one of the first to eleventh aspects, in which the ultrasound image is a moving image including a plurality of frames, in a case in which N and M are natural numbers equal to or larger than 2, the processor displays the first mark in the ultrasound image in a case in which the lesion region is detected from N consecutive frames among the plurality of frames, and displays the second mark in the ultrasound image in a case in which the organ region is detected from M consecutive frames among the plurality of frames, and N is a value smaller than M.

A fifteenth aspect according to the technology of the present disclosure relates to the diagnosis support device according to any one of the first to fourteenth aspects, in which the processor notifies of detection of the lesion region by causing a sound reproduction device to output a sound and/or a vibration generator to generate a vibration in a case in which the lesion region is detected.

A sixteenth aspect according to the technology of the present disclosure relates to the diagnosis support device according to any one of the first to fifteenth aspects, in which the processor displays a plurality of screens including a first screen and a second screen on the display device, displays the ultrasound image on the first screen and the second screen, and separately displays the first mark and the second mark in the ultrasound image on the first screen and in the ultrasound image on the second screen.

A seventeenth aspect according to the technology of the present disclosure relates to the diagnosis support device according to any one of the first to sixteenth aspects, in which the processor detects the lesion region and the organ region from the ultrasound image.

An eighteenth aspect according to the technology of the present disclosure relates to an ultrasound endoscope comprising: the diagnosis support device according to any one of the first to seventeenth aspects; and an ultrasound endoscope body to which the diagnosis support device is connected.

A nineteenth aspect according to the technology of the present disclosure relates to a diagnosis support method comprising: acquiring an ultrasound image; displaying the acquired ultrasound image on a display device; and displaying, in the ultrasound image, a first mark capable of specifying a lesion region detected from the ultrasound image within the ultrasound image and a second mark capable of specifying an organ region detected from the ultrasound image within the ultrasound image, in which the first mark is displayed in a state of being emphasized more than the second mark.

A twentieth aspect according to the technology of the present disclosure relates to a program for causing a computer to execute a process comprising: acquiring an ultrasound image; displaying the acquired ultrasound image on a display device; and displaying, in the ultrasound image, a first mark capable of specifying a lesion region detected from the ultrasound image within the ultrasound image and a second mark capable of specifying an organ region detected from the ultrasound image within the ultrasound image, in which the first mark is displayed in a state of being emphasized more than the second mark.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a configuration of an ultrasound endoscope;

FIG. 4 is a conceptual diagram showing an example of an aspect in which a trained model is generated by training a model using training data;

FIG. 5 is a conceptual diagram showing an example of a processing content of a generation unit;

FIG. 6 is a conceptual diagram showing an example of processing contents of the generation unit and a detection unit;

FIG. 7 is a conceptual diagram showing an example of processing contents in which a control unit generates a mark based on a detection frame;

FIG. 8 is a conceptual diagram showing an example of an aspect in which an ultrasound image to which the mark is assigned is displayed on a screen of a display device;

FIG. 9A is a flowchart showing an example of a flow of diagnosis support processing;

FIG. 9B is a continuation of the flowchart shown in FIG. 9A;

FIG. 10 is a conceptual diagram showing an example of a processing content according to a first modification example;

FIG. 11A is a flowchart showing an example of a flow of diagnosis support processing according to the first modification example;

FIG. 11B is a continuation of the flowchart shown in FIG. 11A;

FIG. 12 is a conceptual diagram showing an example of an aspect in which an ultrasound image to which a first mark is assigned and an ultrasound image to which a second mark is assigned are displayed on separate screens in an endoscope system according to a second modification example; and

FIG. 13 is a conceptual diagram showing an example of an aspect in which a control unit controls a sound reproduction device and a vibration generator in an endoscope system according to a third modification example.

### DETAILED DESCRIPTION

Hereinafter, an example of embodiments of a diagnosis support device, an ultrasound endoscope, a diagnosis support method, and a program according to the technology of the present disclosure will be described with reference to the accompanying drawings.

First, the terms used in the following description will be described.

CPU is an abbreviation for “central processing unit”. GPU is an abbreviation for “graphics processing unit”. TPU is an abbreviation for “tensor processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. PC is an abbreviation for “personal computer”. LAN is an abbreviation for “local area network”. WAN is an abbreviation for “wide area network”. AI is an abbreviation for “artificial intelligence”. BLI is an abbreviation for “blue light imaging”. LCI is an abbreviation for “linked color imaging”. NN is an abbreviation for “neural network”. CNN is an abbreviation for “convolutional neural network”. R-CNN is an abbreviation for “region-based convolutional neural network”. YOLO is an abbreviation for “you only look once”. RNN is an abbreviation for “recurrent neural network”. FCN is an abbreviation for “fully convolutional network”.

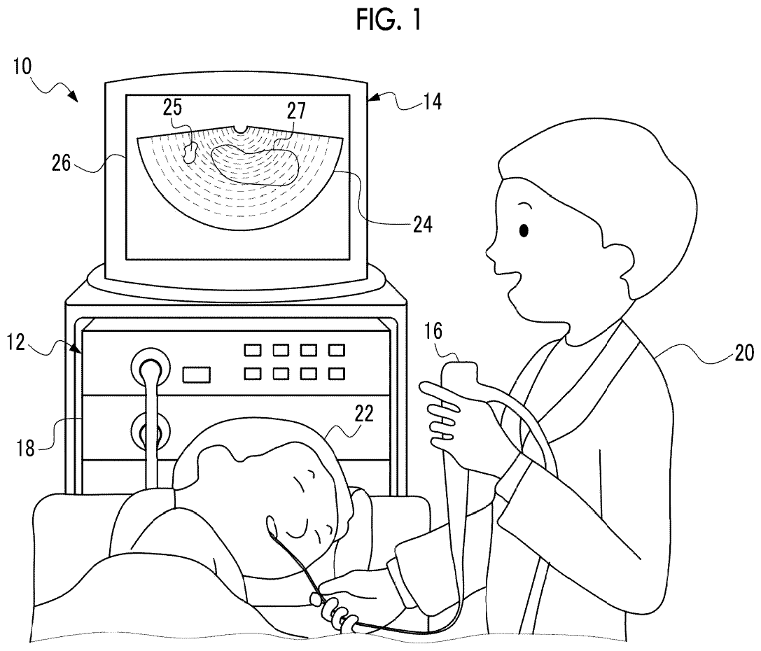

As shown in FIG. 1 as an example, an endoscope system 10 comprises an ultrasound endoscope 12 and a display device 14. The ultrasound endoscope 12 is a convex ultrasound endoscope, and comprises an ultrasound endoscope body 16 and a processing device 18. The ultrasound endoscope 12 is an example of an “ultrasound endoscope” according to the technology of the present disclosure. The processing device 18 is an example of a “diagnosis support device” according to the technology of the present disclosure. The ultrasound endoscope body 16 is an example of an “ultrasound endoscope body” according to the technology of the present disclosure. The display device 14 is an example of a “display device” according to the technology of the present disclosure.

It should be noted that, in the present embodiment, although the convex ultrasound endoscope is shown as an example of the ultrasound endoscope 12, this is merely an example, and the technology of the present disclosure is also implementable in a radial ultrasound endoscope.

The ultrasound endoscope body 16 is used by, for example, a doctor 20. The processing device 18 is connected to the ultrasound endoscope body 16, and transmits and receives various signals to and from the ultrasound endoscope body 16. That is, the processing device 18 outputs the signal to the ultrasound endoscope body 16 to control an operation of the ultrasound endoscope body 16, or executes various types of signal processing on the signal input from the ultrasound endoscope body 16.

The ultrasound endoscope 12 is a device for executing medical care (for example, diagnosis and/or treatment) on a medical care target part (for example, an organ such as a pancreas) in a body of a subject 22, and generates and outputs an ultrasound image 24 indicating an observation target region including the medical care target part.

For example, in a case of observing the observation target region in the body of the subject 22, the doctor 20 inserts the ultrasound endoscope body 16 into the body of the subject 22 from a mouth or a nose of the subject 22 (in the example shown in FIG. 1, the mouth), and emits ultrasound at a position such as a stomach or a duodenum. The ultrasound endoscope body 16 emits the ultrasound to the observation target region in the body of the subject 22 and detects a reflected wave obtained by the reflected ultrasound in the observation target region.

It should be noted that the example shown in FIG. 1 shows an aspect in which an upper gastrointestinal endoscopy is executed, but the technology of the present disclosure is not limited to this, and the technology of the present disclosure can also be applied to a lower gastrointestinal endoscopy, a bronchoscopy, and the like.

The processing device 18 generates the ultrasound image 24 based on the reflected wave detected by the ultrasound endoscope body 16 and outputs the ultrasound image 24 to the display device 14 or the like.

The display device 14 displays various types of information including an image under the control of the processing device 18. Examples of the display device 14 include a liquid-crystal display and an EL display. The ultrasound image 24 generated by the processing device 18 is displayed as a moving image on a screen 26 of the display device 14. The moving image is generated in accordance with a predetermined frame rate (for example, several tens of frames/second) and is displayed on the screen 26. The example shown in FIG. 1 shows an aspect in which the ultrasound image 24 on the screen 26 includes a lesion region 25 indicating a portion corresponding to a lesion and an organ region 27 indicating a portion corresponding to the organ (that is, an aspect in which the lesion and the organ are shown in the ultrasound image 24). The lesion region 25 is an example of a “lesion region” according to the technology of the present disclosure. The organ region 27 is an example of an “organ region” according to the technology of the present disclosure.

It should be noted that the example shown in FIG. 1 shows a form example in which the ultrasound image 24 is displayed on the screen 26 of the display device 14, but this is merely an example, and the ultrasound image 24 may be displayed on a display device (for example, a display of a tablet terminal) other than the display device 14. The ultrasound image 24 may be stored in a computer-readable non-transitory storage medium (for example, a flash memory, an HDD, and/or a magnetic tape).

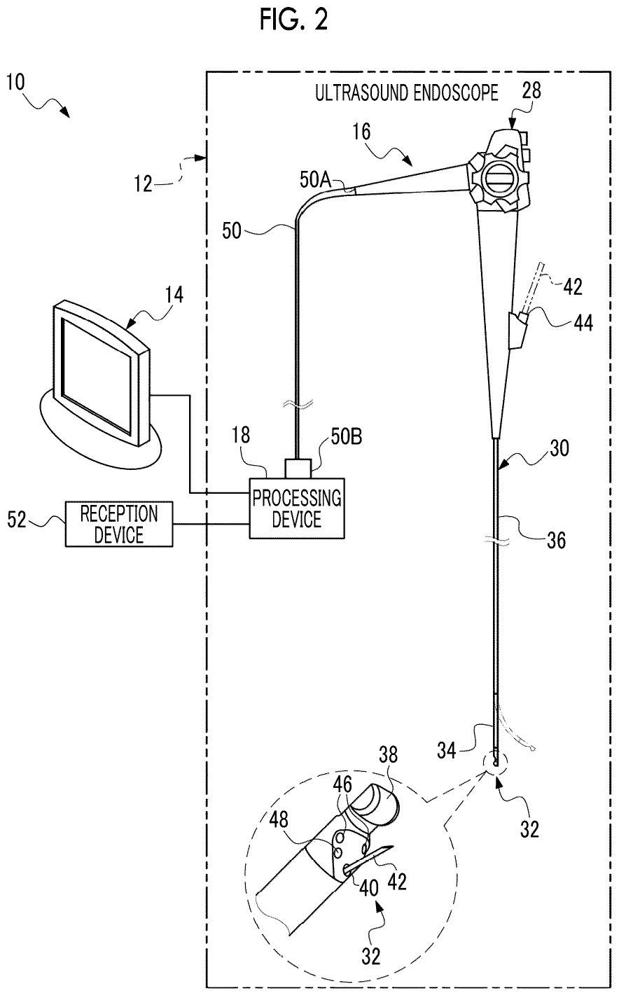

As shown in FIG. 2 as an example, the ultrasound endoscope body 16 comprises an operating part 28 and an insertion part 30. The insertion part 30 is formed in a tubular shape. The insertion part 30 includes a distal end part 32, a bendable part 34, and a flexible part 36. The distal end part 32, the bendable part 34, and the flexible part 36 are disposed in an order of the distal end part 32, the bendable part 34, and the flexible part 36 from a distal end side to a base end side of the insertion part 30. The flexible part 36 is formed of a material having a long and flexible shape and connects the operating part 28 and the bendable part 34. The bendable part 34 is partially bent or rotated about an axial center of the insertion part 30 by operating the operating part 28. As a result, the insertion part 30 is sent to a back side of a luminal organ while being bent in accordance with a shape of the luminal organ (for example, a shape of a duodenal pathway) or being rotated about an axis of the insertion part 30.

The distal end part 32 is provided with an ultrasound probe 38 and a treatment tool opening 40. The ultrasound probe 38 is provided on the distal end side of the distal end part 32. The ultrasound probe 38 is a convex ultrasound probe, and emits the ultrasound and receives the reflected wave obtained by the emitted ultrasound being reflected in the observation target region.

The treatment tool opening 40 is formed on the base end side of the distal end part 32 with respect to the ultrasound probe 38. The treatment tool opening 40 is an opening for allowing a treatment tool 42 to protrude from the distal end part 32. A treatment tool insertion port 44 is formed at the operating part 28, and the treatment tool 42 is inserted into the insertion part 30 through the treatment tool insertion port 44. The treatment tool 42 passes through the insertion part 30 and protrudes from the treatment tool opening 40 to the outside of the ultrasound endoscope body 16. The treatment tool opening 40 also functions as a suction port for suctioning blood, internal waste, and the like.

In the example shown in FIG. 2, a puncture needle is shown as the treatment tool 42. It should be noted that this is merely an example, and the treatment tool 42 may be forceps and/or a sheath.

In the example shown in FIG. 2, an illumination device 46 and a camera 48 are provided in the distal end part 32. The illumination device 46 emits light. Examples of a type of light emitted from the illumination device 46 include visible light (for example, white light), invisible light (for example, near-infrared light), and/or special light. Examples of the special light include light for BLI and/or light for LCI.

The camera 48 images the inside of the luminal organ using an optical method. Examples of the camera 48 include a CMOS camera. The CMOS camera is merely an example, and another type of camera, such as a CCD camera, may be used. It should be noted that the image obtained by being captured by the camera 48 is displayed on the display device 14, is displayed on a display device (for example, a display of a tablet terminal) other than the display device 14, or is stored in a storage medium (for example, a flash memory, an HDD, and/or a magnetic tape).

The ultrasound endoscope 12 comprises the processing device 18 and a universal cord 50. The universal cord 50 has a base end part 50A and a distal end part 50B. The base end part 50A is connected to the operating part 28. The distal end part 50B is connected to the processing device 18. That is, the ultrasound endoscope body 16 and the processing device 18 are connected to each other via the universal cord 50.

The endoscope system 10 comprises a reception device 52. The reception device 52 is connected to the processing device 18. The reception device 52 receives an instruction from a user. Examples of the reception device 52 include an operation panel having a plurality of hard keys and/or a touch panel, a keyboard, a mouse, a trackball, a foot switch, a smart device, and/or a microphone.

The processing device 18 executes various types of signal processing or transmits and receives various signals to and from the ultrasound endoscope body 16 and the like in response to the instruction received by the reception device 52. For example, the processing device 18 emits the ultrasound to the ultrasound probe 38 in response to the instruction received by the reception device 52, generates the ultrasound image 24 (see FIG. 1) based on the reflected wave received by the ultrasound probe 38, and outputs the generated ultrasound image 24.

The display device 14 is also connected to the processing device 18. The processing device 18 controls the display device 14 in response to the instruction received by the reception device 52. As a result, for example, the ultrasound image 24 generated by the processing device 18 is displayed on the screen 26 of the display device 14 (see FIG. 1).

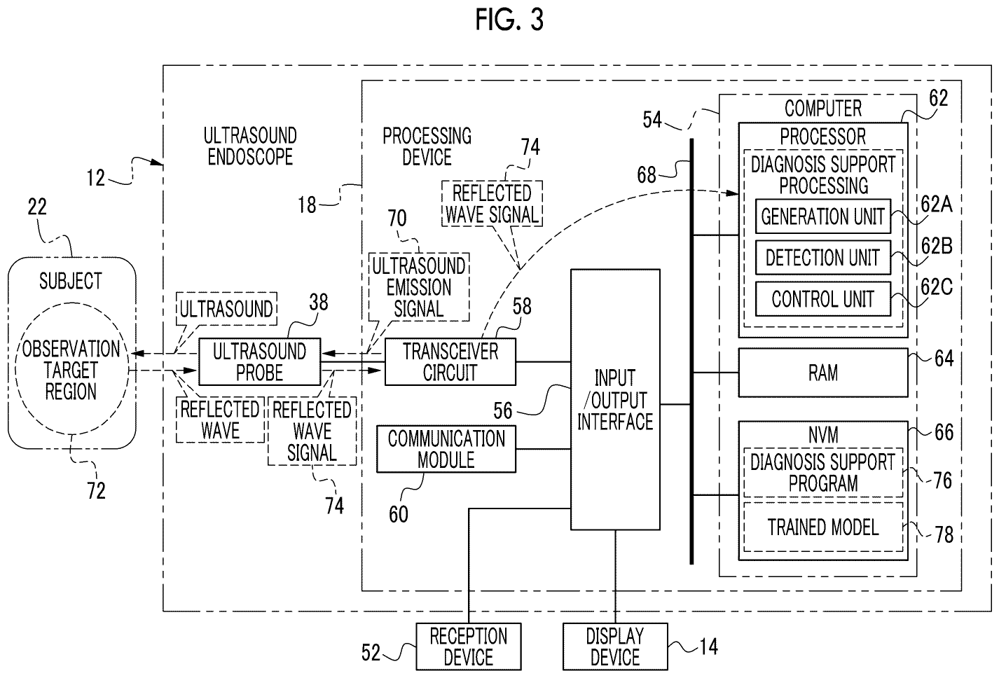

As shown in FIG. 3 as an example, the processing device 18 comprises a computer 54, an input/output interface 56, a transceiver circuit 58, and a communication module 60. The computer 54 is an example of a “computer” according to the technology of the present disclosure.

The computer 54 comprises a processor 62, a RAM 64, and an NVM 66. The input/output interface 56, the processor 62, the RAM 64, and the NVM 66 are connected to a bus 68.

The processor 62 controls the entire processing device 18. For example, the processor 62 includes a CPU and a GPU, and the GPU is operated under the control of the CPU, and is mainly responsible for executing image processing. It should be noted that the processor 62 may be one or more CPUs integrated with a GPU function or may be one or more CPUs not integrated with the GPU function. The processor 62 may include a multi-core CPU or a TPU. The processor 62 is an example of a “processor” according to the technology of the present disclosure.

The RAM 64 is a memory that temporarily stores information, and is used as a work memory by the processor 62. The NVM 66 is a non-volatile storage device that stores various programs and various parameters. Examples of the NVM 66 include a flash memory (for example, an EEPROM) and/or an SSD. It should be noted that the flash memory and the SSD are merely examples, and the NVM 66 may be other non-volatile storage devices such as an HDD, or may be a combination of two or more types of non-volatile storage devices.

The reception device 52 is connected to the input/output interface 56, and the processor 62 acquires the instruction received by the reception device 52 via the input/output interface 56 and executes processing in response to the acquired instruction.

The transceiver circuit 58 is connected to the input/output interface 56. The transceiver circuit 58 generates an ultrasound emission signal 70 having a pulse waveform to output the ultrasound emission signal 70 to the ultrasound probe 38 in response to an instruction from the processor 62. The ultrasound probe 38 converts the ultrasound emission signal 70 input from the transceiver circuit 58 into the ultrasound and emits the ultrasound to an observation target region 72 of the subject 22. The ultrasound probe 38 receives the reflected wave obtained by reflecting the ultrasound emitted from the ultrasound probe 38 from the observation target region 72, converts the reflected wave into a reflected wave signal 74, which is an electric signal, and outputs the reflected wave signal 74 to the transceiver circuit 58. The transceiver circuit 58 digitizes the reflected wave signal 74 input from the ultrasound probe 38 and outputs the digitized reflected wave signal 74 to the processor 62 via the input/output interface 56. The processor 62 generates the ultrasound image 24 (see FIG. 1) showing an aspect of the observation target region 72 based on the reflected wave signal 74 input from the transceiver circuit 58 via the input/output interface 56.

Although not shown in FIG. 3, the illumination device 46 (see FIG. 2) is also connected to the input/output interface 56. The processor 62 controls the illumination device 46 via the input/output interface 56 to change the type of the light emitted from the illumination device 46 or to adjust an amount of the light. In addition, although not shown in FIG. 3, the camera 48 (see FIG. 2) is also connected to the input/output interface 56. The processor 62 controls the camera 48 via the input/output interface 56 or acquires the image obtained by imaging the inside of the body of the subject 22 using the camera 48 via the input/output interface 56.

The communication module 60 is connected to the input/output interface 56. The communication module 60 is an interface including a communication processor, an antenna, and the like. The communication module 60 is connected to a network (not shown) such as a LAN or a WAN, and controls communication between the processor 62 and an external device.

The display device 14 is connected to the input/output interface 56, and the processor 62 controls the display device 14 via the input/output interface 56 such that various types of information are displayed on the display device 14.

The reception device 52 is connected to the input/output interface 56, and the processor 62 acquires the instruction received by the reception device 52 via the input/output interface 56 and executes processing in response to the acquired instruction.

The NVM 66 stores a diagnosis support program 76 and a trained model 78. The diagnosis support program 76 is an example of a “program” according to the technology of the present disclosure. The trained model 78 is a trained model having a data structure used for processing of detecting the lesion and the organ from the ultrasound image 24.

The processor 62 executes diagnosis support processing by reading out the diagnosis support program 76 from the NVM 66 and executing the readout diagnosis support program 76 on the RAM 64. The diagnosis support processing is processing of detecting the lesion and the organ from the observation target region 72 using an AI method, and supporting a diagnosis performed by the doctor 20 (see FIG. 1) based on a detection result. The detection of the lesion and the organ using the AI method is implemented by using the trained model 78.

The processor 62 executes the diagnosis support processing to detect a portion corresponding to the lesion and a portion corresponding to the organ from the ultrasound image 24 (see FIG. 1) in accordance with the trained model 78, thereby detecting the lesion and the organ from the observation target region 72. The diagnosis support processing is implemented by the processor 62 operating as a generation unit 62A, a detection unit 62B, and a control unit 62C in accordance with the diagnosis support program 76 executed on the RAM 64.

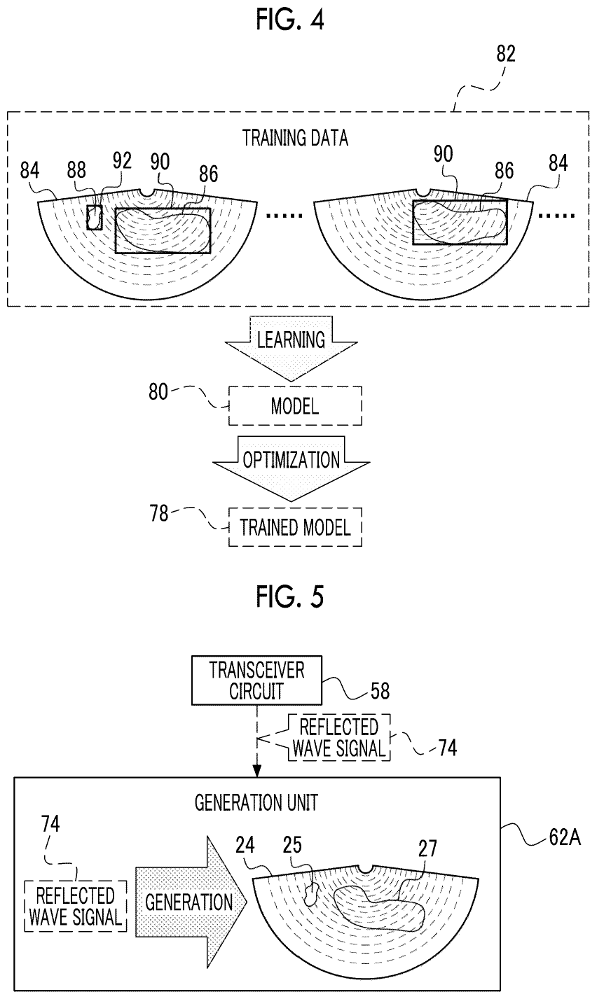

As shown in FIG. 4 as an example, the trained model 78 is generated by training a model 80 that has not been trained. The model 80 is trained using training data 82. The training data 82 includes a plurality of different ultrasound images 84. For example, the ultrasound image 84 is an ultrasound image generated by the convex ultrasound endoscope, similarly to the ultrasound image 24 (see FIG. 1).

The plurality of ultrasound images 84 include an ultrasound image in which the organ (for example, the pancreas) is shown, an ultrasound image in which the lesion is shown, and an ultrasound image in which the organ and the lesion are shown. The example shown in FIG. 4 shows an aspect in which an organ region 86 indicating the portion corresponding to the organ is included in the ultrasound image 84 and an aspect in which a lesion region 88 indicating the portion corresponding to the lesion is included in the ultrasound image 84.

Examples of the model 80 include a mathematical model using an NN. Examples of a type of the NN include a YOLO, an R-CNN, and an FCN. In addition, the NN used in the model 80 may be the YOLO, the R-CNN, or a combination of the FCN and an RNN. The RNN is suitable for learning of a plurality of images obtained in time series. It should be noted that the type of the NN described here is merely an example, and other types of the NNs capable of detecting an object by learning an image may be used.

In the example shown in FIG. 4, an organ annotation 90 is assigned to the organ region 86 in the ultrasound image 84. The organ annotation 90 is information capable of specifying a position of the organ region 86 in the ultrasound image 84 (for example, information including a plurality of coordinates capable of specifying a position of a rectangular frame that circumscribes the organ region 86). Here, for convenience of description, the information capable of specifying the position of the organ region 86 in the ultrasound image 84 is shown as an example of the organ annotation 90, but this is merely an example. For example, the organ annotation 90 may include other types of information capable of specifying the organ shown in the ultrasound image 84, such as information capable of specifying the type of the organ shown in the ultrasound image 84.

In the example shown in FIG. 4, a lesion annotation 92 is assigned to the lesion region 88. The lesion annotation 92 is information capable of specifying a position of the lesion region 88 in the ultrasound image 84 (for example, information including a plurality of coordinates capable of specifying a position of a rectangular frame that circumscribes the lesion region 88). Here, for convenience of description, the information capable of specifying the position of the lesion region 88 in the ultrasound image 84 is shown as an example of the lesion annotation 92, but this is merely an example. For example, the lesion annotation 92 may include other types of information capable of specifying the lesion shown in the ultrasound image 84, such as information capable of specifying the type of the lesion shown in the ultrasound image 84.

It should be noted that, hereinafter, for convenience of description, in a case in which it is not necessary to distinguish between the organ annotation 90 and the lesion annotation 92, the organ annotation 90 and the lesion annotation 92 will be referred to as an “annotation” without reference numerals. In addition, hereinafter, for convenience of description, the processing using the trained model 78 will be described as processing that is actively executed with the trained model 78 as a main subject. That is, for convenience of description, the trained model 78 will be described as having a function of executing processing on input information and outputting a processing result. In addition, hereinafter, for convenience of description, a part of processing of training the model 80 will also be described as processing that is actively executed with the model 80 as a main subject. That is, for convenience of description, the model 80 will be described as having a function of executing processing on input information and outputting a processing result.

The training data 82 is input to the model 80. That is, each ultrasound image 84 is input to the model 80. The model 80 predicts the position of the organ region 86 and/or the position of the lesion region 88 from the input ultrasound image 84, and outputs a prediction result. The prediction result includes information capable of specifying the position predicted by the model 80 as the position of the organ region 86 in the ultrasound image 84 and/or information capable of specifying the position predicted by the model 80 as the position of the lesion region 88 in the ultrasound image 84.

Here, examples of the information capable of specifying the position predicted by the model 80 as the position of the organ region 86 in the ultrasound image 84 include information including a plurality of coordinates capable of specifying a position of a bounding box surrounding a region predicted as a position at which the organ region 86 is present (that is, the position of the bounding box in the ultrasound image 84). Examples of the information capable of specifying the position predicted by the model 80 as the position of the lesion region 88 in the ultrasound image 84 include information including a plurality of coordinates capable of specifying a position of a bounding box surrounding a region predicted as a position at which the lesion region 88 is present (that is, the position of the bounding box in the ultrasound image 84).

The model 80 is adjusted in accordance with an error between the annotation assigned to the ultrasound image 84 input to the model 80 and the prediction result output from the model 80. That is, the model 80 is optimized by adjusting a plurality of optimization variables (for example, a plurality of connection weights and a plurality of offset values) in the model 80 such that the error is minimized, and thereby the trained model 78 is generated. That is, the data structure of the trained model 78 is obtained by training the model 80 using the plurality of different ultrasound images 84 to which the annotations are assigned.

According to a known technology in the related art, a result of the detection of the lesion region 25 (see FIG. 1) via the trained model 78 and a result of the detection of the organ region 27 (see FIG. 1) via the trained model 78 are visualized by being displayed on the screen 26 or the like as marks such as detection frames. The marks such as the detection frames indicate the positions of the lesion region 25 and the organ region 27. A frequency with which the lesion region 25 is displayed on the screen 26 or the like (in other words, a frequency with which the lesion region 25 appears) is lower than a frequency with which the organ region 27 is displayed (in other words, a frequency with which the organ region 27 appears). This means that, in a case in which the diagnosis using the ultrasound image 24 is performed by the doctor 20, a probability of overlooking of the lesion region 25 is higher than a probability of overlooking of the organ region 27.

In addition, in a case in which the mark such as the detection frame assigned to the organ region 27 and the mark such as the detection frame assigned to the lesion region 25 are displayed on the screen 26 or the like in a mixed state, the presence of the mark such as the detection frame assigned to the organ region 27 may hinder the diagnosis. This can also increase the probability of the overlooking of the lesion region 25.

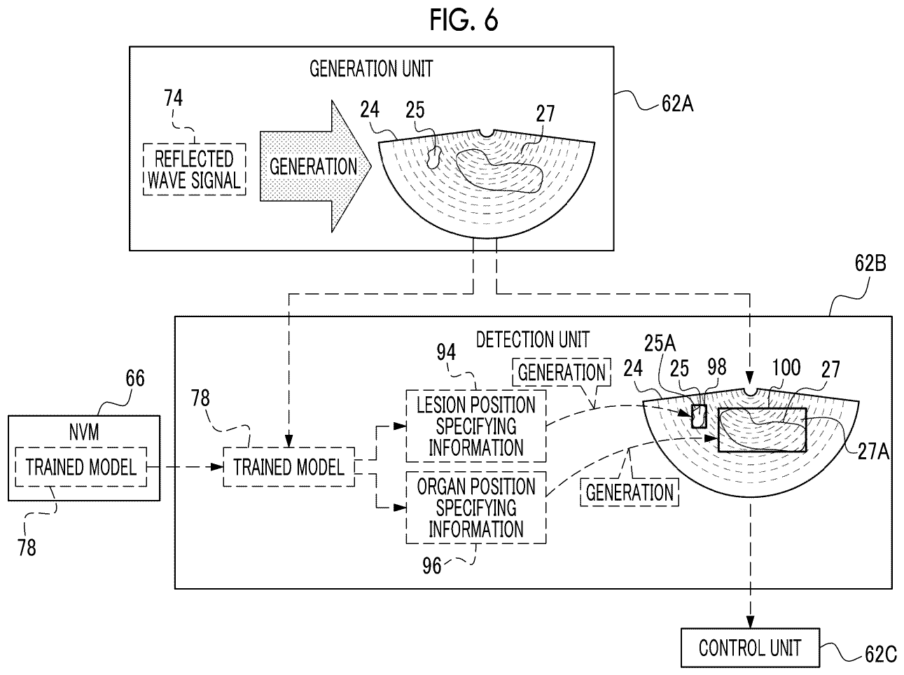

Therefore, in view of the above-described circumstances, in the processing device 18 according to the present embodiment, as an example, the diagnosis support processing is executed as shown in FIGS. 5 to 9B. Hereinafter, an example of the diagnosis support processing will be described in detail.

As shown in FIG. 5 as an example, the generation unit 62A acquires the reflected wave signal 74 from the transceiver circuit 58 and generates the ultrasound image 24 based on the acquired reflected wave signal 74, thereby acquiring the ultrasound image 24. The ultrasound image 24 is an example of an “ultrasound image” according to the technology of the present disclosure.

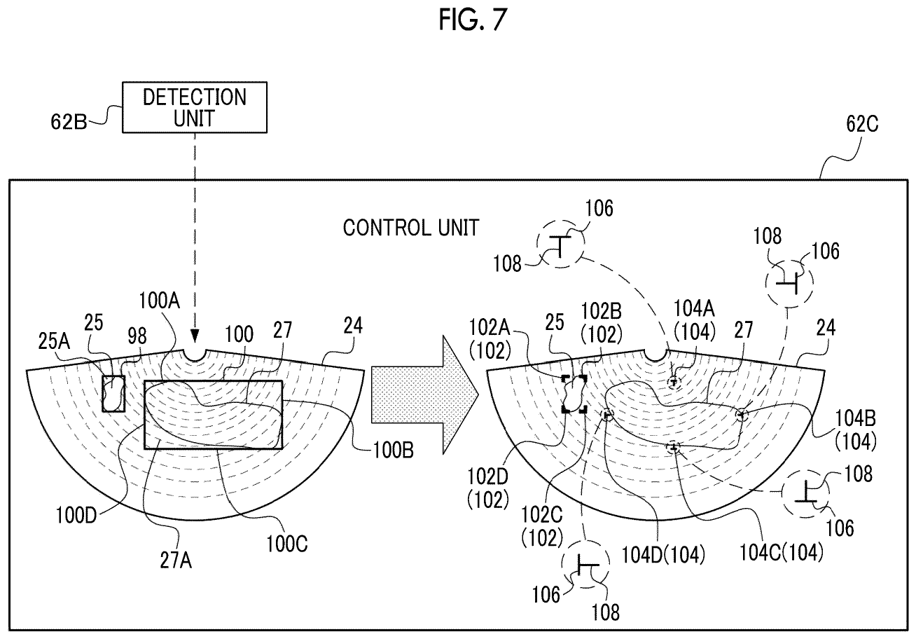

As shown in FIG. 6 as an example, the detection unit 62B detects the lesion by detecting the lesion region 25 from the ultrasound image 24 generated by the generation unit 62A in accordance with the trained model 78. That is, the detection unit 62B determines the presence or absence of the lesion region 25 in the ultrasound image 24 in accordance with the trained model 78, and generates lesion position specifying information 94 for specifying the position of the lesion region 25 (for example, information including a plurality of coordinates for specifying the position of the lesion region 25) in a case in which the lesion region 25 is present in the ultrasound image 24. Here, in a case in which the processing of detecting the lesion via the detection unit 62B is described with the trained model 78 as the main subject, in a case in which the ultrasound image 24 generated by the generation unit 62A is input, the trained model 78 determines the presence or absence of the lesion region 25 in the input ultrasound image 24. The trained model 78 outputs the lesion position specifying information 94 in a case in which it is determined that the lesion region 25 is present in the ultrasound image 24 (that is, in a case in which the lesion shown in the ultrasound image 24 is detected).

The detection unit 62B detects the organ by detecting the organ region 27 from the ultrasound image 24 generated by the generation unit 62A in accordance with the trained model 78. That is, the detection unit 62B determines the presence or absence of the organ region 27 in the ultrasound image 24 in accordance with the trained model 78, and generates organ position specifying information 96 for specifying the position of the organ region 27 (for example, information including a plurality of coordinates for specifying the position of the organ region 27) in a case in which the organ region 27 is present in the ultrasound image 24. Here, in a case in which the processing of detecting the organ via the detection unit 62B is described with the trained model 78 as the main subject, in a case in which the ultrasound image 24 generated by the generation unit 62A is input, the trained model 78 determines the presence or absence of the organ region 27 in the input ultrasound image 24. The trained model 78 outputs the organ position specifying information 96 in a case in which it is determined that the organ region 27 is present in the ultrasound image 24 (that is, in a case in which the organ shown in the ultrasound image 24 is detected).

The detection unit 62B generates detection frames 98 and 100 and superimposes the generated detection frames 98 and 100 on the ultrasound image 24, thereby assigning the detection frames 98 and 100 to the ultrasound image 24.

The detection frame 98 is a rectangular frame corresponding to a bounding box (for example, a bounding box having a highest reliability score for the lesion region 25) used in a case in which the trained model 78 detects the lesion region 25 from the ultrasound image 24. That is, the detection frame 98 is a frame that surrounds a range 25A in which the lesion region 25 detected by the trained model 78 is present. The range 25A is a rectangular range and is defined by the detection frame 98. In the example shown in FIG. 6, a rectangular frame that circumscribes the lesion region 25 is shown as an example of the detection frame 98. It should be noted that the rectangular frame that circumscribes the lesion region 25 is merely an example, and the technology of the present disclosure is implementable even in a case in which a frame that does not circumscribe the lesion region 25 is used.

The detection unit 62B assigns the detection frame 98 to the ultrasound image 24 corresponding to the lesion position specifying information 94 output from the trained model 78 (that is, the ultrasound image 24 input to the trained model 78 for outputting the lesion position specifying information 94) in accordance with the lesion position specifying information 94. That is, the detection unit 62B superimposes the detection frame 98 on the ultrasound image 24 corresponding to the lesion position specifying information 94 output from the trained model 78 to surround the lesion region 25, thereby assigning the detection frame 98 to the ultrasound image 24.

The detection frame 100 is a rectangular frame corresponding to a bounding box (for example, a bounding box having a highest reliability score for the organ region 27) used in a case in which the trained model 78 detects the organ region 27 from the ultrasound image 24. That is, the detection frame 100 is a frame that surrounds the organ region 27 detected by the trained model 78. That is, the detection frame 100 is a frame that surrounds a range 27A in which the organ region 27 detected by the trained model 78 is present. The range 27A is a rectangular range and is defined by the detection frame 100. In the example shown in FIG. 6, a rectangular frame that circumscribes the organ region 27 is shown as an example of the detection frame 100. It should be noted that the rectangular frame that circumscribes the organ region 27 is merely an example, and the technology of the present disclosure is implementable even in a case in which a frame that does not circumscribe the organ region 27 is used.

The detection unit 62B assigns the detection frame 100 to the ultrasound image 24 corresponding to the organ position specifying information 96 output from the trained model 78 (that is, the ultrasound image 24 input to the trained model 78 for outputting the organ position specifying information 96) in accordance with the organ position specifying information 96. That is, the detection unit 62B superimposes the detection frame 100 on the ultrasound image 24 corresponding to the organ position specifying information 96 output from the trained model 78 to surround the organ region 27, thereby assigning the detection frame 100 to the ultrasound image 24.

In the present embodiment, the detection frame 98 is an example of a “first rectangular frame” according to the technology of the present disclosure. The range 25A is an example of a “first range” according to the technology of the present disclosure. The detection frame 100 is an example of a “second rectangular frame” according to the technology of the present disclosure. The range 27A is an example of a “second range” according to the technology of the present disclosure. It should be noted that, hereinafter, for convenience of description, in a case in which it is not necessary to distinguish between the detection frames 98 and 100, the detection frames 98 and 100 will be referred to as a “detection frame” without reference numerals.

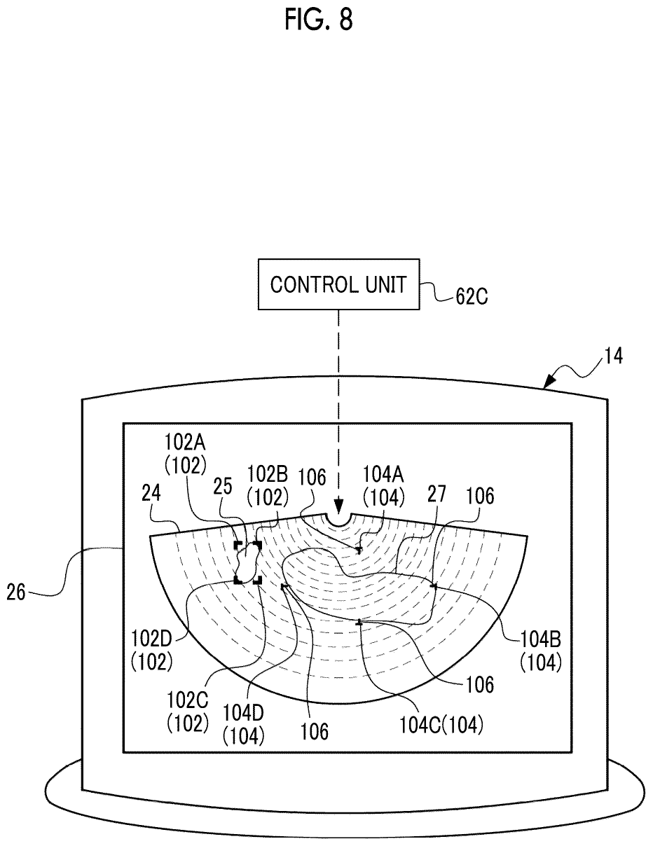

As shown in FIG. 7 as an example, the control unit 62C acquires the ultrasound image 24 in which the detection result is reflected, from the detection unit 62B. The example shown in FIG. 7 shows an aspect in which the ultrasound image 24 to which the detection frames 98 and 100 are assigned is acquired and processed by the control unit 62C.

In the ultrasound image 24, the detection frame 98 surrounds the lesion region 25 in a rectangular shape as seen in front view. In addition, in the ultrasound image 24, the detection frame 100 surrounds the organ region 27 in a rectangular shape as seen in front view. Here, the front view refers to, for example, a state in which the screen 26 is viewed from a front side in a case in which the ultrasound image 24 is displayed on the screen 26 of the display device 14.

The control unit 62C generates a first mark 102 based on the detection frame 98. The first mark 102 is a mark capable of specifying the lesion region 25 detected from the ultrasound image 24 by the detection unit 62B in the ultrasound image 24. The first mark 102 is formed to be capable of specifying an outer edge of the range 25A.

The first mark 102 is composed of four images. In the example shown in FIG. 7, the four images refer to L-shaped pieces 102A to 102D. In each of the L-shaped pieces 102A to 102D, a part of the detection frame 98 is imaged. That is, each of the L-shaped pieces 102A to 102D is a mark in which a part of the detection frame 98 is formed in a visually specifiable manner. In the example shown in FIG. 7, the L-shaped pieces 102A to 102D are formed to have the same shape and the same size.

In the example shown in FIG. 7, positions of the L-shaped pieces 102A to 102D correspond to positions of four corners of the detection frame 98. Each of the L-shaped pieces 102A to 102D is formed in a shape of a corner of the detection frame 98. That is, each of the L-shaped pieces 102A to 102D is formed in an L-shape. As described above, a position of the range 25A in the ultrasound image 24 can be specified by assigning the L-shaped pieces 102A to 102D to the four corners of the detection frame 98. The L-shaped pieces 102A to 102D are examples of a “plurality of first images” according to the technology of the present disclosure.

The control unit 62C generates a second mark 104 based on the detection frame 100. The second mark 104 is a mark capable of specifying the organ region 27 detected from the ultrasound image 24 by the detection unit 62B within the ultrasound image 24. The second mark 104 is formed to be capable of specifying an outer edge of the range 27A.

The second mark 104 is composed of four images. In the example shown in FIG. 7, the four images constituting the second mark 104 refer to T-shaped pieces 104A to 104D. In each of the T-shaped pieces 104A to 104D, a part of the detection frame 100 is imaged. That is, each of the T-shaped pieces 104A to 104D is a mark in which a part of the detection frame 100 is formed in a visually specifiable manner. In the example shown in FIG. 7, respective positions of the T-shaped pieces 104A to 104D correspond to positions of respective center portions of sides 100A to 100D constituting the detection frame 100. Each of the T-shaped pieces 104A to 104D is formed in a T-shape. In the example shown in FIG. 7, the T-shaped pieces 104A to 104D are formed to have the same shape and the same size. Each of the T-shaped pieces 104A to 104D consists of straight lines 106 and 108. One end of the straight line 108 is located at a midpoint of the straight line 106, and the straight line 108 is disposed perpendicular to the straight line 106.

The straight line 106 of the T-shaped piece 104A is formed at a position parallel to the side 100A and overlapping with the side 100A. The straight line 108 of the T-shaped piece 104A extends from a midpoint of the side 100A to a lower side as seen in front view. The straight line 106 of the T-shaped piece 104B is formed at a position parallel to the side 100B and overlapping with the side 100B. The straight line 108 of the T-shaped piece 104B extends from a midpoint of the side 100B to a left side as seen in front view. The straight line 106 of the T-shaped piece 104C is formed at a position parallel to the side 100C and overlapping with the side 100C. The straight line 108 of the T-shaped piece 104C extends from a midpoint of the side 100C to an upper side as seen in front view. The straight line 106 of the T-shaped piece 104D is formed at a position parallel to the side 100D and overlapping with the side 100D. The straight line 108 of the T-shaped piece 104D extends from a midpoint of the side 100D to a right side as seen in front view.

As described above, a position of the range 27A in the ultrasound image 24 can be specified by assigning the T-shaped pieces 104A to 104D to the respective center portions of the sides 100A to 100D of the detection frame 100. The T-shaped pieces 104A to 104D are examples of a “plurality of second images” according to the technology of the present disclosure.

In the example shown in FIG. 7, the first mark 102 is formed in a state of being emphasized more than the second mark 104. Here, the state of being emphasized means a state in which the first mark 102 is more visually conspicuous than the second mark 104 in a case in which the first mark 102 and the second mark 104 are displayed on the screen 26 in a mixed state. In the example shown in FIG. 7, the first mark 102 is formed by a thicker line than the second mark 104, and the L-shaped pieces 102A to 102D are formed in larger sizes than the T-shaped pieces 104A to 104D. As a result, the first mark 102 is in a state of being emphasized more than the second mark 104. Hereinafter, for convenience of description, in a case in which it is not necessary to distinguish between the first mark 102 and the second mark 104, the first mark 102 and the second mark 104 will be referred to as a “mark” without reference numerals.

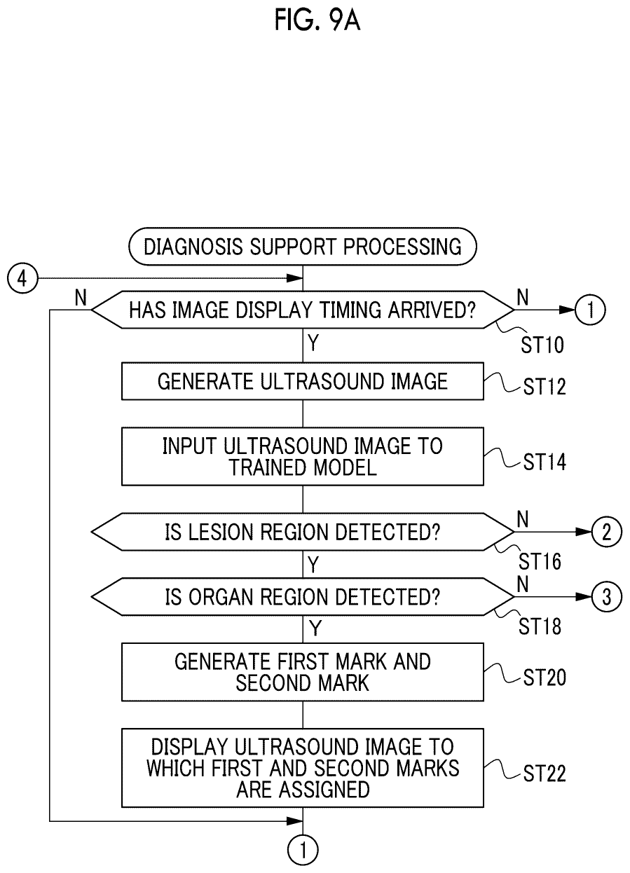

As an example, as shown in FIG. 8, the control unit 62C displays the ultrasound image 24 generated by the generation unit 62A on the display device 14. In this case, in a case in which the lesion and the organ are not detected by the detection unit 62B, the control unit 62C displays the ultrasound image 24 to which the mark is not assigned on the screen 26 of the display device 14. In addition, in a case in which the lesion and/or the organ is detected by the detection unit 62B, the control unit 62C displays the ultrasound image 24 to which the mark is assigned on the screen 26 of the display device 14. The first mark 102 is displayed on the screen 26 at a position corresponding to the lesion region 25 in the ultrasound image 24. That is, the L-shaped pieces 102A to 102D are displayed to surround the lesion region 25. In other words, the L-shaped pieces 102A to 102D are displayed to be capable of specifying the outer edge of the range 25A (see FIGS. 6 and 7). As a result, the position of the lesion region 25 in the ultrasound image 24 can be visually understood.

In addition, the second mark 104 is displayed on the screen 26 at a position corresponding to the organ region 27 in the ultrasound image 24. That is, the T-shaped pieces 104A to 104D are displayed to surround the organ region 27. In other words, the T-shaped pieces 104A to 104D are displayed to be capable of specifying the outer edge of the range 27A (see FIGS. 6 and 7).

Further, on the screen 26, the first mark 102 is displayed in a state of being emphasized more than the second mark 104. As a result, the position of the lesion region 25 and the position of the organ region 27 can be visually discriminated.

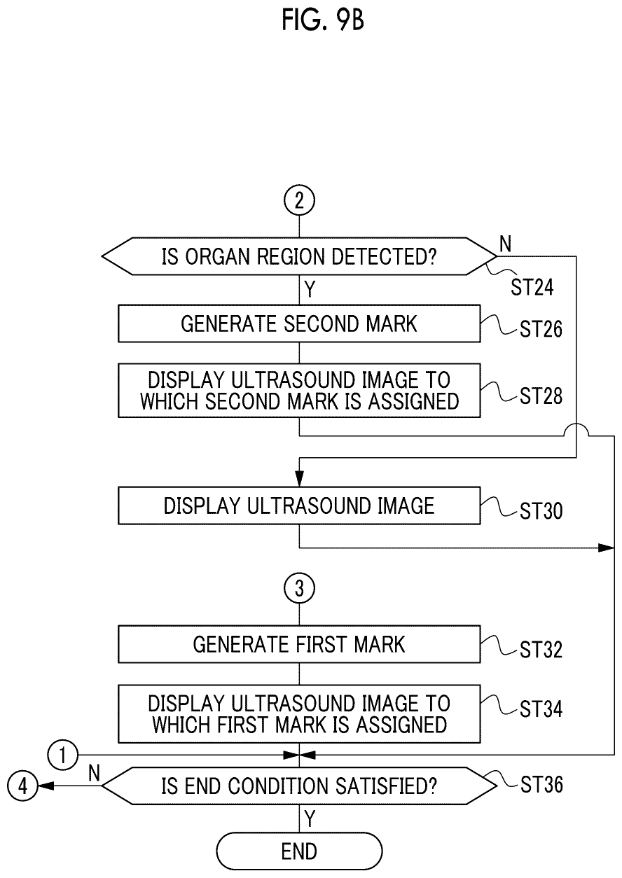

Next, an operation of the endoscope system 10 will be described with reference to FIGS. 9A and 9B.

FIGS. 9A and 9B show an example of a flow of the diagnosis support processing executed by the processor 62 of the processing device 18 on the condition that the diagnosis using the endoscope system 10 is started (for example, the emission of the ultrasound via the ultrasound endoscope 12 is started). The flow of the diagnosis support processing shown in FIGS. 9A and 9B is an example of a “diagnosis support method” according to the technology of the present disclosure.

In the diagnosis support processing shown in FIG. 9A, first, in step ST10, the generation unit 62A determines whether or not an image display timing has arrived. The image display timing is, for example, a timing divided by a time interval defined by the reciprocal of the frame rate. In step ST10, in a case in which the image display timing has not arrived, a negative determination is made, and the diagnosis support processing proceeds to step ST36 shown in FIG. 9B. In step ST10, in a case in which the image display timing has arrived, an affirmative determination is made, and the diagnosis support processing proceeds to step ST12.

In step ST12, the generation unit 62A generates the ultrasound image 24 based on the reflected wave signal 74 input from the transceiver circuit 58 (see FIG. 5). After the processing of step ST12 is executed, the diagnosis support processing proceeds to step ST14.

In step ST14, the detection unit 62B inputs the ultrasound image 24 generated in step ST12 to the trained model 78. After the processing of step ST14 is executed, the diagnosis support processing proceeds to step ST16.

In step ST16, the detection unit 62B determines whether or not the lesion region 25 is included in the ultrasound image 24 input to the trained model 78 in step ST14, by using the trained model 78. In a case in which the lesion region 25 is included in the ultrasound image 24, the trained model 78 outputs the lesion position specifying information 94 (see FIG. 6).

In step ST16, in a case in which the lesion region 25 is not included in the ultrasound image 24, a negative determination is made, and the diagnosis support processing proceeds to step ST24 shown in FIG. 9B. In step ST16, in a case in which the lesion region 25 is included in the ultrasound image 24, an affirmative determination is made, and the diagnosis support processing proceeds to step ST18.

In step ST18, the detection unit 62B determines whether or not the organ region 27 is included in the ultrasound image 24 input to the trained model 78 in step ST14, by using the trained model 78. In a case in which the organ region 27 is included in the ultrasound image 24, the trained model 78 outputs the organ position specifying information 96 (see FIG. 6).

In step ST18, in a case in which the organ region 27 is not included in the ultrasound image 24, a negative determination is made, and the diagnosis support processing proceeds to step ST32 shown in FIG. 9B. In step ST18, in a case in which the organ region 27 is included in the ultrasound image 24, an affirmative determination is made, and the diagnosis support processing proceeds to step ST20.

In step ST20, the control unit 62C generates the first mark 102 (see FIG. 7) based on the lesion position specifying information 94 (see FIG. 6). Specifically, the control unit 62C generates the detection frame 98 based on the lesion position specifying information 94 (see FIG. 6), and generates the first mark 102 based on the detection frame 98 (see FIG. 7). In addition, the control unit 62C generates the second mark 104 (see FIG. 7) based on the organ position specifying information 96 (see FIG. 6). Specifically, the control unit 62C generates the detection frame 100 based on the organ position specifying information 96 (see FIG. 6), and generates the second mark 104 based on the detection frame 100 (see FIG. 7). The first mark 102 and the second mark 104 generated in this manner are superimposed on the ultrasound image 24 generated in step ST12, thereby being assigned to the ultrasound image 24. After the processing of step ST20 is executed, the diagnosis support processing proceeds to step ST22.

In step ST22, the control unit 62C displays the ultrasound image 24 on which the first mark 102 and the second mark 104 are superimposed, on the screen 26 of the display device 14 (see FIG. 8). The first mark 102 is displayed in a state of being emphasized more than the second mark 104. After the processing of step ST22 is executed, the diagnosis support processing proceeds to step ST36 shown in FIG. 9B.

In step ST24 shown in FIG. 9B, the detection unit 62B determines whether or not the organ region 27 is included in the ultrasound image 24 input to the trained model 78 in step ST14, by using the trained model 78. In a case in which the organ region 27 is included in the ultrasound image 24, the trained model 78 outputs the organ position specifying information 96 (see FIG. 6).

In step ST24, in a case in which the organ region 27 is not included in the ultrasound image 24, a negative determination is made, and the diagnosis support processing proceeds to step ST30. In step ST24, in a case in which the organ region 27 is included in the ultrasound image 24, an affirmative determination is made, and the diagnosis support processing proceeds to step ST26.

In step ST26, the control unit 62C generates the second mark 104 (see FIG. 7) based on the organ position specifying information 96 (see FIG. 6). The second mark 104 is superimposed on the ultrasound image 24 generated in step ST12, thereby being assigned to the ultrasound image 24. After the processing of step ST26 is executed, the diagnosis support processing proceeds to step ST28.

In step ST28, the control unit 62C displays the ultrasound image 24 on which the second mark 104 is superimposed, on the screen 26 of the display device 14. After the processing of step ST28 is executed, the diagnosis support processing proceeds to step ST36.

In step ST30, the control unit 62C displays the ultrasound image 24 generated in step ST12 on the screen 26 of the display device 14. After the processing of step ST30 is executed, the diagnosis support processing proceeds to step ST36.

In step ST32, the control unit 62C generates the first mark 102 (see FIG. 7) based on the lesion position specifying information 94 (see FIG. 6). The first mark 102 is superimposed on the ultrasound image 24 generated in step ST12, thereby being assigned to the ultrasound image 24. After the processing of step ST32 is executed, the diagnosis support processing proceeds to step ST34.

In step ST34, the control unit 62C displays the ultrasound image 24 on which the first mark 102 is superimposed, on the screen 26 of the display device 14. After the processing of step ST34 is executed, the diagnosis support processing proceeds to step ST36.

In step ST36, the control unit 62C determines whether or not a condition for ending the diagnosis support processing (hereinafter, referred to as a “diagnosis support end condition”) is satisfied. Examples of the diagnosis support end condition include a condition in which an instruction to end the diagnosis support processing is received by the reception device 52. In step ST36, in a case in which the diagnosis support end condition is not satisfied, a negative determination is made, and the diagnosis support processing proceeds to step ST10 shown in FIG. 9A. In step ST36, in a case in which the diagnosis support end condition is satisfied, an affirmative determination is made, and the diagnosis support processing ends.

As described above, in the endoscope system 10, in a case in which the lesion region 25 is detected, the first mark 102 that surrounds the lesion region 25 is generated (see FIG. 7), and in a case in which the organ region 27 is detected, the second mark 104 that surrounds the organ region 27 is generated (see FIG. 7). The ultrasound image 24 on which the first mark 102 and the second mark 104 are superimposed is displayed on the screen 26 of the display device 14. The first mark 102 is displayed in a state of being emphasized more than the second mark 104. As a result, it is easy for the doctor 20 to visually discriminate the lesion region 25 and the organ region 27. Since the second mark 104 has a lower visual expression intensity than the first mark 102, the overlooking of the first mark 102 due to the second mark 104 being too conspicuous is suppressed. Therefore, the overlooking of the lesion region 25 can be suppressed in the diagnosis using the ultrasound image 24.

The first mark 102 is the mark capable of specifying the outer edge of the range 25A in which the lesion region 25 is present. Therefore, the outer edge of the range 25A in which the lesion region 25 is present can be visually recognized by the doctor 20 from the ultrasound image 24.

The range 25A in which the lesion region 25 is present is defined by the detection frame 98 that is the rectangular frame that surrounds the lesion region 25. Therefore, the range 25A in which the lesion region 25 is present can be processed in units of the detection frame 98 that is the rectangular frame.

The detection frame 98 is the rectangular frame that circumscribes the lesion region 25. Therefore, the doctor 20 can specify the range 25A in which the lesion region 25 is present with higher accuracy than in a case in which a rectangular frame that does not circumscribe the lesion region 25 (for example, a rectangular frame that is located further out than the lesion region 25) is used.

The first mark 102 is the mark in which a part of the detection frame 98 is formed in a visually specifiable manner. Therefore, the range 25A in which the lesion region 25 is present can be visually recognized by the doctor 20 in units of the detection frame 98.

The first mark 102 consists of the L-shaped pieces 102A to 102D disposed at the four corners of the detection frame 98. Therefore, as compared with a case in which the entire detection frame 98 is displayed, it is possible to reduce the number of elements that hinder the observation in a case in which the doctor 20 observes the ultrasound image 24.

The second mark 104 is the mark capable of specifying the outer edge of the range 27A in which the organ region 27 is present. Therefore, the outer edge of the range 27A in which the organ region 27 is present can be visually recognized by the doctor 20 from the ultrasound image 24.

The range 27A in which the organ region 27 is present is defined by the detection frame 100 that is the rectangular frame that surrounds the organ region 27. Therefore, the range 27A in which the organ region 27 is present can be processed in units of the detection frame 100 that is the rectangular frame.

The detection frame 100 is the rectangular frame that circumscribes the organ region 27. Therefore, the doctor 20 can specify the range 27A in which the organ region 27 is present with higher accuracy than in a case in which a rectangular frame that does not circumscribe the organ region 27 (for example, a rectangular frame that is located further out than the organ region 27) is used.

The second mark 104 is the mark in which a part of the detection frame 100 is formed in a visually specifiable manner. Therefore, the range 27A in which the organ region 27 is present can be visually recognized by the doctor 20 in units of the detection frame 100.

The second mark 104 consists of the T-shaped pieces 104A to 104D disposed at the respective center portions of the four sides of the detection frame 100. Therefore, as compared with a case in which the entire detection frame 100 is displayed, it is possible to reduce the number of elements that hinder the observation in a case in which the doctor 20 observes the ultrasound image 24.

In addition, in a case in which the T-shaped pieces 104A to 104D are displayed as the second mark 104, a distance between the adjacent T-shaped pieces among the T-shaped pieces 104A to 104D is shorter than in a case in which the marks are disposed at the four corners of the detection frame 100, and thus it is possible to make it difficult to lose sight of the second mark 104 (that is, the T-shaped pieces 104A to 104D) as compared with a case in which the marks are disposed at the four corners of the detection frame 100. In particular, although it is easier to lose sight of each mark disposed at each of the four corners of the detection frame 100 as the organ region 27 becomes larger and as the marks disposed at the four corners of the detection frame 100 become smaller, even under such conditions, it is possible to make it difficult to lose sight of the second mark 104 (that is, the T-shaped pieces 104A to 104D) as compared with a case in which the marks are disposed at the four corners of the detection frame 100.

Further, as shown in FIGS. 7 and 8 as an example, an intersection of a straight line connecting a midpoint of the T-shaped piece 104A and a midpoint of the T-shaped piece 104C and a straight line connecting a midpoint of the T-shaped piece 104B and a midpoint of the T-shaped piece 104D (hereinafter, simply referred to as an “intersection”) is a point included in the center portion of the range 27A in which the organ region 27 is present. A direction of the straight line 108 of the T-shaped piece 104A, a direction of the straight line 108 of the T-shaped piece 104B, a direction of the straight line 108 of the T-shaped piece 104C, and a direction of the straight line 108 of the T-shaped piece 104D indicate a position of the intersection. Therefore, the doctor 20 can visually estimate the position of the center portion of the range 27A in which the organ region 27 is present, from the positions of the T-shaped pieces 104A to 104D.

The processor 62 detects the lesion region 25 and the organ region 27. Therefore, the lesion region 25 detected by the processor 62 can be visually recognized by the doctor 20 through the first mark 102, and the organ region 27 detected by the processor 62 can be visually recognized by the doctor 20 through the second mark 104.

### First Modification Example

In the embodiment described above, the form example has been described in which the detection result of the lesion region 25 via the detection unit 62B and the detection result of the organ region 27 via the detection unit 62B are displayed as the marks in units of frames (that is, each time the ultrasound image 24 is generated), but the technology of the present disclosure is not limited to this. For example, the control unit 62C may display the first mark 102 in the ultrasound image 24 in a case in which the lesion region 25 is detected from N consecutive frames among a plurality of frames, and display the second mark 104 in the ultrasound image 24 in a case in which the organ region 27 is detected from M consecutive frames among the plurality of frames. Here, N and M mean natural numbers satisfying a magnitude relationship of “N<M”, and the plurality of frames mean a plurality of ultrasound images 24 (for example, a plurality of ultrasound images 24 constituting a moving image) along a time series.

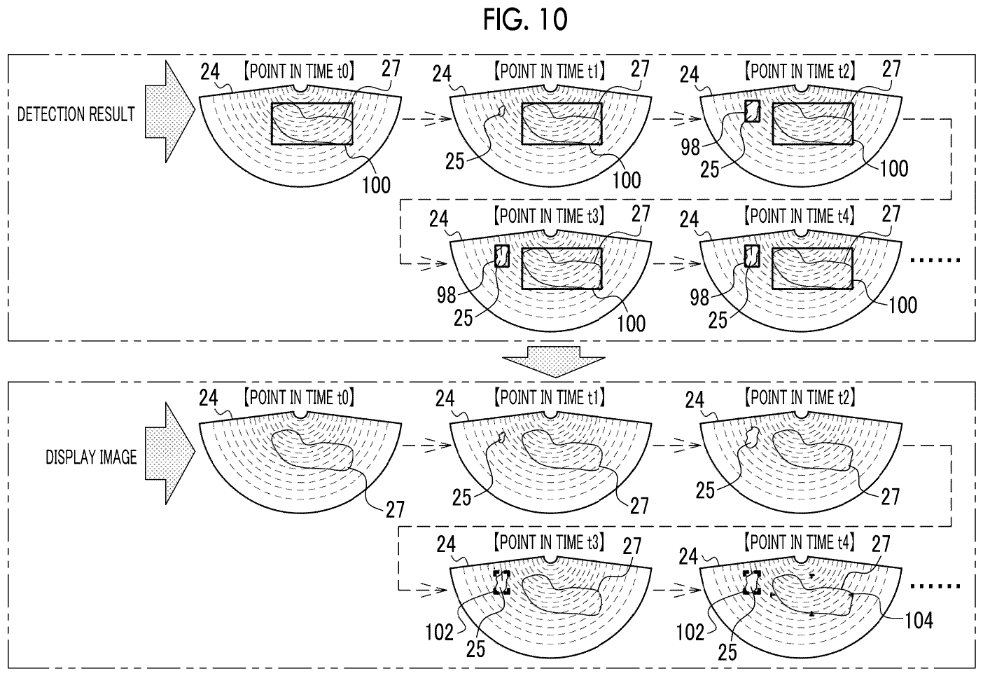

Here, in a case in which N is set to “2” and M is set to “5”, as shown in FIG. 10 as an example, the first mark 102 is displayed in the ultrasound image 24 on the condition that the lesion region 25 is detected from two consecutive frames, and the second mark 104 is displayed in the ultrasound image 24 on the condition that the organ region 27 is detected from five consecutive frames.

The example shown in FIG. 10 shows an aspect in which the second mark 104 is displayed at a point in time t4 in a case in which the organ region 27 is consecutively detected from a point in time t0 to the point in time t4. In addition, the example shown in FIG. 10 shows an aspect in which the first mark 102 is displayed at a point in time t3 in a case in which the lesion region 25 is consecutively detected at a point in time t2 and the point in time t3, and the first mark 102 is displayed at the point in time t4 in a case in which the lesion region 25 is consecutively detected at the point in time t3 and the point in time t4.

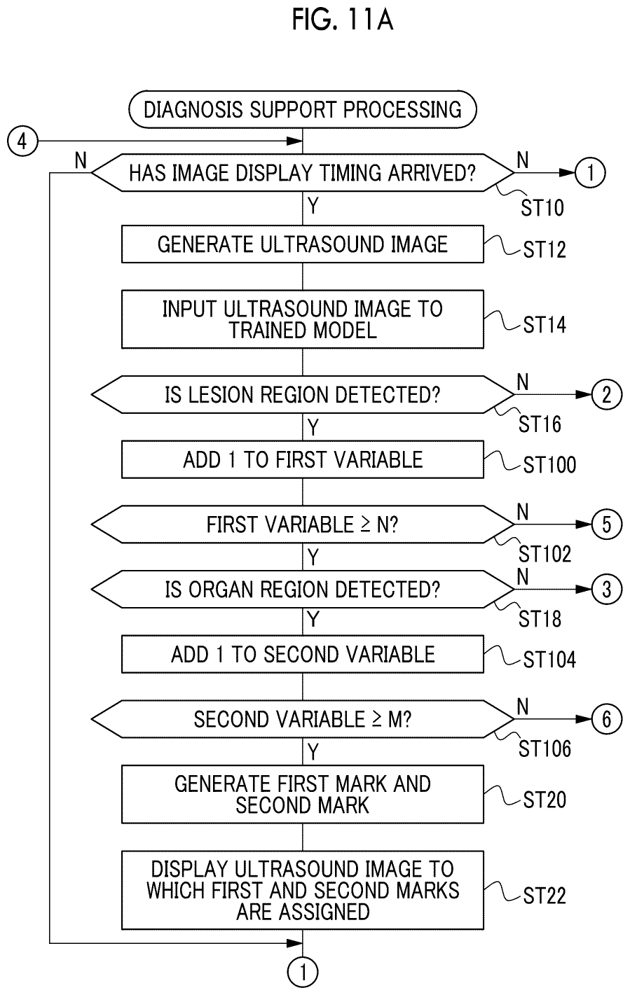

FIGS. 11A and 11B show an example of a flow of diagnosis support processing according to the first modification example. The flowcharts shown in FIGS. 11A and 11B are different from the flowcharts shown in FIGS. 9A and 9B in that processing of step ST100 to processing of step ST116 are added.

The processing of step ST100 and the processing of step ST102 are provided between the processing of step ST16 and the processing of step ST18. The processing of step ST104 and the processing of step ST106 are provided between the processing of step ST18 and the processing of step ST20. The processing of step ST108 is provided prior to the processing of step ST24, and is executed in a case in which a negative determination is made in step ST16. The processing of step ST110 and the processing of step ST112 are provided between the processing of step ST24 and the processing of step ST26. The processing of step ST114 is provided prior to the processing of step ST30, and is executed in a case in which a negative determination is made in step ST24. The processing of step ST116 is provided prior to the processing of step ST32, and is executed in a case in which a negative determination is made in step ST18.

In the diagnosis support processing shown in FIG. 11A, in step ST100, the detection unit 62B adds 1 to a first variable of which an initial value is “0”. After the processing of step ST100 is executed, the diagnosis support processing proceeds to step ST102.

In step ST102, the detection unit 62B determines whether or not the first variable is equal to or larger than N. In step ST102, in a case in which the first variable is smaller than N, a negative determination is made, and the diagnosis support processing proceeds to step ST24 shown in FIG. 11B. In step ST102, in a case in which the first variable is equal to or larger than N, an affirmative determination is made, and the diagnosis support processing proceeds to step ST18.

In step ST104, the detection unit 62B adds 1 to a second variable of which an initial value is “0”. After the processing of step ST102 is executed, the diagnosis support processing proceeds to step ST106.

In step ST106, the detection unit 62B determines whether or not the second variable is equal to or larger than M. In step ST106, in a case in which the second variable is smaller than M, a negative determination is made, and the diagnosis support processing proceeds to step ST32 shown in FIG. 11B. In step ST106, in a case in which the second variable is equal to or larger than M, an affirmative determination is made, and the diagnosis support processing proceeds to step ST20.

In step ST108 shown in FIG. 11B, the detection unit 62B resets the first variable. That is, the first variable is returned to the initial value thereof. After the processing of step ST108 is executed, the diagnosis support processing proceeds to step ST24.

In step ST110, the detection unit 62B adds 1 to the second variable. After the processing of step ST110 is executed, the diagnosis support processing proceeds to step ST112.

In step ST112, the detection unit 62B determines whether or not the second variable is equal to or larger than M. In step ST112, in a case in which the second variable is smaller than M, a negative determination is made, and the diagnosis support processing proceeds to step ST30. In step ST112, in a case in which the second variable is equal to or larger than M, an affirmative determination is made, and the diagnosis support processing proceeds to step ST26.

In step ST114, the detection unit 62B resets the second variable. That is, the second variable is returned to the initial value thereof. After the processing of step ST114 is executed, the diagnosis support processing proceeds to step ST30.

In step ST116, the detection unit 62B resets the second variable. After the processing of step ST116 is executed, the diagnosis support processing proceeds to step ST32.

As described above, in the first modification example, in a case in which the lesion region 25 is detected from the N consecutive frames among the plurality of frames, the first mark 102 is displayed in the ultrasound image 24. Therefore, since the detection result with a higher reliability degree is visualized as the first mark 102, compared with a case in which the ultrasound image 24 in which the detection result is reflected for each frame is displayed, it is possible to prevent a situation in which the doctor 20 erroneously discriminates a region, which is not the lesion region 25, as the lesion region 25. In addition, in a case in which the organ region 27 is detected from the M consecutive frames among the plurality of frames, the second mark 104 is displayed in the ultrasound image 24. Therefore, since the detection result with a higher reliability degree is visualized as the second mark 104, compared with a case in which the ultrasound image 24 in which the detection result is reflected for each frame is displayed, it is possible to prevent a situation in which the doctor 20 erroneously discriminates a region, which is not the organ region 27, as the organ region 27.

In addition, in the first modification example, the natural numbers satisfying the magnitude relationship of N<M are used as N and M. In the first modification example, since the first mark 102 is displayed on the condition that the lesion region 25 is detected from the N consecutive frames, for example, a frequency of visualizing the detection result of the lesion region 25 as the first mark 102 is higher than that in a case in which the first mark 102 is visualized on the condition that the lesion region 25 is detected from the M consecutive frames. Therefore, it is possible to reduce a risk of the overlooking of the lesion region 25. In addition, in the first modification example, since the second mark 104 is displayed on the condition that the organ region 27 is detected from the M consecutive frames, for example, a frequency of visualizing the detection result of the organ region 27 as the second mark 104 is lower than that in a case in which the second mark 104 is visualized on the condition that the organ region 27 is detected from the N consecutive frames. Therefore, it is possible to prevent a situation in which the second mark 104 hinders the diagnosis due to the high frequency display of the second mark 104.

It should be noted that, in the first modification example, although “2” is described as N and “5” is described as M, this is merely an example, and N and M need only be natural numbers equal to or larger than 2, and N and M are preferably natural numbers satisfying the magnitude relationship of “N<M”.

### Second Modification Example

In the embodiment described above, the form example has been described in which only the ultrasound image 24 to which the mark is assigned is displayed on the screen 26, but the technology of the present disclosure is not limited to this. For example, the ultrasound image 24 to which only the first mark 102 is assigned out of the first mark 102 and the second mark 104 and the ultrasound image 24 to which only the second mark 104 is assigned out of the first mark 102 and the second mark 104 may be separately displayed.

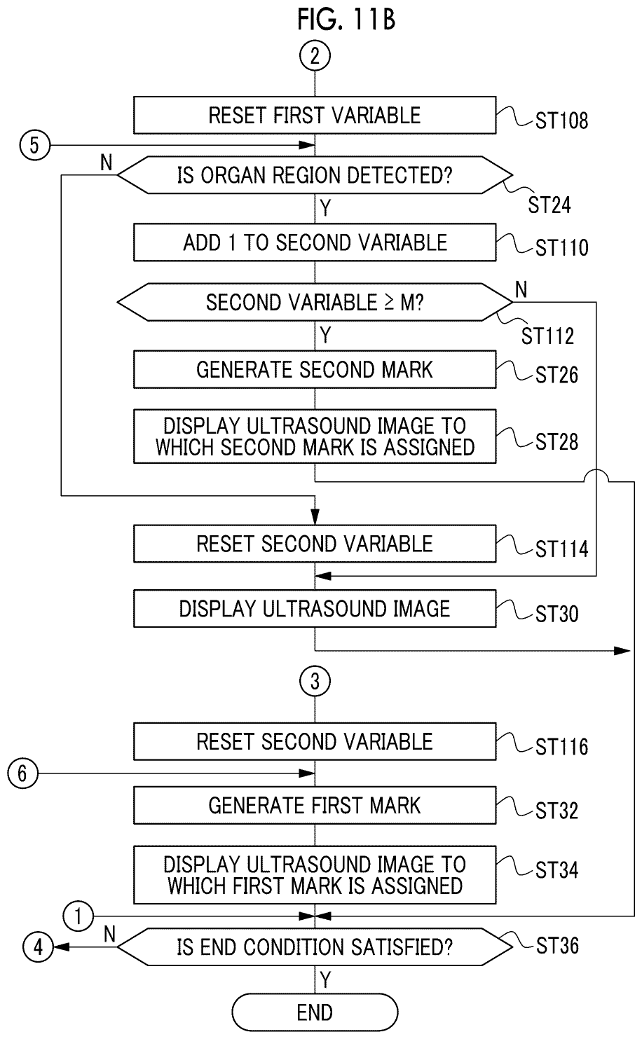

In this case, for example, as shown in FIG. 12, the control unit 62C displays a first screen 26A and a second screen 26B side by side on the display device 14. Then, the control unit 62C displays the ultrasound image 24 to which only the first mark 102 is assigned out of the first mark 102 and the second mark 104, on the first screen 26A. In addition, the control unit 62C displays the ultrasound image 24 to which only the second mark 104 is assigned out of the first mark 102 and the second mark 104, on the second screen 26B. As a result, the visibility of the ultrasound image 24 is increased as compared with a case in which the first mark 102 and the second mark 104 are mixed in one ultrasound image 24. It should be noted that the first screen 26A is an example of a “first screen” according to the technology of the present disclosure, and the second screen 26B is an example of a “second screen” according to the technology of the present disclosure.

The example shown in FIG. 12 shows a form example in which the control unit 62C displays the first screen 26A and the second screen 26B side by side on the display device 14, but this is merely an example, and the control unit 62C may display a screen corresponding to the first screen 26A on the display device 14 and display a screen corresponding to the second screen 26B on a display device other than the display device 14.

In addition, the ultrasound image 24 to which the first mark 102 and the second mark 104 are assigned, the ultrasound image 24 to which only the first mark 102 is assigned out of the first mark 102 and the second mark 104, and the ultrasound image 24 to which only the second mark 104 is assigned out of the first mark 102 and the second mark 104 may be selectively displayed on the screen 26 (see FIG. 1) depending on a given condition. Here, a first example of the given condition is a condition in which the instruction from the user is received by the reception device 52. A second example of the given condition is a condition in which at least one designated lesion is detected. A third example of the given condition is a condition in which at least one designated organ is detected.

### Third Modification Example

In the embodiment described above, the form example has been described in which the doctor 20 understands the detection of the lesion region 25 by visually recognizing the first mark 102 displayed in the ultrasound image 24, and the doctor 20 understands the detection of the organ region 27 by visually recognizing the second mark 104 displayed in the ultrasound image 24, but the technology of the present disclosure is not limited to this. For example, the doctor 20 may be notified of the detection of the lesion region 25 and/or the organ region 27 by outputting a sound and/or generating a vibration.

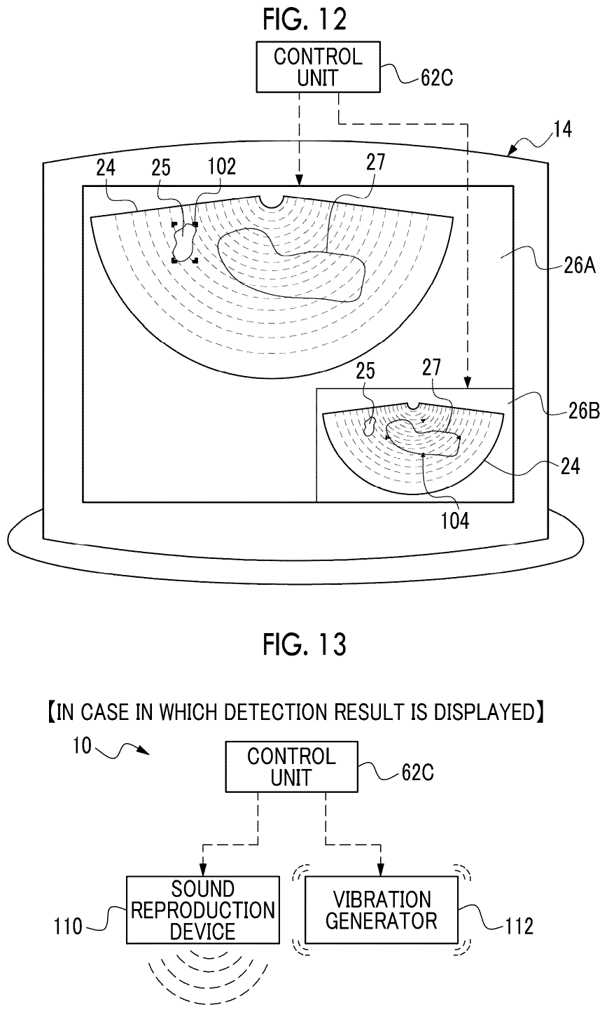

In this case, for example, as shown in FIG. 13, the endoscope system 10 comprises a sound reproduction device 110 and a vibration generator 112, and the control unit 62C controls the sound reproduction device 110 and the vibration generator 112. For example, in a case in which the detection unit 62B detects the lesion region 25, the control unit 62C reproduces a sound representing information indicating the detection of the lesion region 25. In addition, in a case in which the detection unit 62B detects the organ region 27, the control unit 62C reproduces a sound representing information indicating the detection of the organ region 27. In addition, in a case in which the lesion region 25 is detected by the detection unit 62B, the control unit 62C generates a vibration representing information indicating the detection of the lesion region 25. Further, in a case in which the organ region 27 is detected by the detection unit 62B, the control unit 62C generates a vibration representing information indicating the detection of the organ region 27. For example, the vibration generator 112 is worn by the doctor 20 in a state of being in contact with a body of the doctor 20, and the doctor 20 perceives the vibration generated by the vibration generator 112 to understand the detection of the lesion region 25 and/or the organ region 27.

In this way, in a case in which the lesion region 25 is detected, notification of the detection of the lesion region 25 is issued by causing the sound reproduction device 110 to reproduce the sound or causing the vibration generator 112 to generate the vibration, so that the risk of the overlooking of the lesion region 25 shown in the ultrasound image 24 can be reduced.

In addition, the control unit 62C may cause the vibration generator 112 to change the magnitude of the vibration and/or an interval of the vibration generation among a case in which the lesion region 25 is detected, a case in which the organ region 27 is detected, and a case in which both the lesion region 25 and the organ region 27 are detected. In addition, the magnitude of the vibration and/or the interval of the vibration generation may be changed depending on the type of the detected lesion, or the magnitude of the vibration and/or the interval of the vibration generation may be changed depending on the type of the detected organ.

It should be noted that, in the third modification example, although the form example has been described in which either the sound is reproduced or the vibration is generated in a case in which the organ region 27 is detected, the sound need not be reproduced or the vibration need not be generated in a case in which the organ region 27 is detected.

### Other Modification Examples

In the embodiment described above, the L-shaped pieces 102A to 102D are described as the first mark 102, but the technology of the present disclosure is not limited to this. For example, a pair of first images may be used in which positions corresponding to opposite corners of four corners of the detection frame 98 can be specified. Examples of the pair of first images include a combination of the L-shaped pieces 102A and 102C and a combination of the L-shaped pieces 102B and 102D. The L-shaped pieces 102A to 102D are merely examples, and pieces having a shape other than the L-shape, such as an I-shape, may be used. The detection frame 98 itself may be used as the first mark 102. A mark having a shape in which a part of the detection frame 98 is missing may be used as the first mark 102. As described above, the first mark 102 need only be a mark in which the position of the lesion region 25 in the ultrasound image 24 can be specified and at least a part of the detection frame 98 is formed in a visually specifiable manner.

In the embodiment described above, the rectangular frame is described as the detection frame 98, but this is merely an example, and a frame having another shape may be used.

In the embodiment described above, the T-shaped pieces 104A to 104D are described as the second marks 104, but the technology of the present disclosure is not limited to this. For example, a pair of second images may be used in which positions corresponding to opposite sides of four sides of the detection frame 100 can be specified. Examples of the pair of second images include a combination of the T-shaped pieces 104A and 104C and a combination of the T-shaped pieces 104B and 104D. The T-shaped pieces 104A to 104D are merely examples, and pieces having a shape other than the T-shape, such as an I-shape, may be used. The detection frame 100 itself may be used as the second mark 104. A mark having a shape in which a part of the detection frame 100 is missing may be used as the second mark 104. As described above, the second mark 104 need only be a mark in which the position of the organ region 27 in the ultrasound image 24 can be specified and at least a part of the detection frame 100 is formed in a visually specifiable manner.

In the embodiment described above, the rectangular frame is described as the detection frame 100, but this is merely an example, and a frame having another shape may be used.

In the embodiment described above, the first mark 102 is emphasized more than the second mark 104 by making the line of the first mark 102 thicker than the line of the second mark 104 and making the sizes of the L-shaped pieces 102A to 102D larger than the sizes of the T-shaped pieces 104A to 104D, but this is merely an example. For example, the second mark 104 may be displayed to be lighter than the first mark 102. The first mark 102 may be displayed in a chromatic color and the second mark 104 may be displayed in an achromatic color. The first mark 102 may be displayed in a more conspicuous line type than the second mark 104. As described above, any display aspect may be used as long as the first mark 102 is displayed in a display aspect of being emphasized more than the second mark 104.

In the embodiment described above, the form example has been described in which the lesion region 25 and the organ region 27 are detected using the AI method (that is, the form example in which the lesion region 25 and the organ region 27 are detected in accordance with the trained model 78), but the technology of the present disclosure is not limited to this, and the lesion region 25 and the organ region 27 may be detected using a non-AI method. Examples of a detection method using the non-AI method include a detection method using template matching.

In the embodiment described above, the form example has been described in which the lesion region 25 and the organ region 27 are detected in accordance with the trained model 78, but the lesion region 25 and the organ region 27 may be detected in accordance with separate trained models. In this case, for example, the lesion region 25 need only be detected in accordance with a trained model obtained by training a model through learning specialized in the detection of the lesion region 25, and the organ region 27 need only be detected in accordance with a trained model obtained by training a model through learning specialized in the detection of the organ region 27.

In the embodiment described above, the ultrasound endoscope 12 has been described as an example, but the technology of the present disclosure can also be applied to an extracorporeal ultrasound diagnostic device.

In the embodiment described above, the form example has been described in which the ultrasound image 24 and the mark, which are generated by the processing device 18, are displayed on the screen 26 of the display device 14, but the ultrasound image 24 to which the mark is assigned may be transmitted to various devices such as a server, a PC, and/or a tablet terminal and stored in memories of the various devices. In addition, the ultrasound image 24 to which the mark is assigned may be recorded in a report. In addition, the detection frames 98 and/or 100 may also be stored in the memories of the various devices or may be recorded in the report. In addition, the lesion position specifying information 94 and/or the organ position specifying information 96 may also be stored in the memories of the various devices or may be recorded in the report. It is preferable that the ultrasound image 24, the mark, the lesion position specifying information 94, the organ position specifying information 96, the detection frame 98, and/or the detection frame 100 is stored in the memories or recorded in the report for each subject 22.

In the embodiment described above, the form example has been described in which the diagnosis support processing is executed by the processing device 18, but the technology of the present disclosure is not limited to this. The diagnosis support processing may be executed by the processing device 18 and at least one device provided outside the processing device 18, or may be executed only by at least one device (for example, an auxiliary processing device that is connected to the processing device 18 and that is used to expand the functions of the processing device 18) provided outside the processing device 18.

Examples of the at least one device provided outside the processing device 18 include a server. The server may be implemented by cloud computing. The cloud computing is merely an example, and network computing, such as fog computing, edge computing, or grid computing, may be used. In addition, the server described as the at least one device provided outside the processing device 18 is merely an example, and may be at least one PC and/or at least one mainframe instead of the server, or may be at least one server, at least one PC, and/or at least one mainframe.

In the embodiment described above, the doctor 20 perceives the presence or absence of the lesion and the position of the lesion, but the doctor 20 may perceive the type of the lesion and/or the degree of progress of the lesion. In this case, the model 80 need only be trained using the ultrasound image 24 in a state in which the lesion annotation 92 includes information capable of specifying the type of the lesion and/or the degree of progress of the lesion.

In the embodiment described above, the doctor 20 perceives the presence or absence of the organ and the position of the organ, but the doctor 20 may perceive the type of the organ and the like. In this case, the model 80 need only be trained using the ultrasound image 24 in a state in which the organ annotation 90 includes information capable of specifying the type of the organ and the like.

In the embodiment described above, the form example has been described in which the detection of the lesion and the detection of the organ are executed by the processing device 18, but the detection of the lesion and/or the detection of the organ may be executed by a device (for example, a server or a PC) other than the processing device 18.

In the embodiment described above, the form example has been described in which the NVM 66 stores the diagnosis support program 76, but the technology of the present disclosure is not limited to this. For example, the diagnosis support program 76 may be stored in a portable storage medium, such as an SSD or a USB memory. The storage medium is a non-transitory computer-readable storage medium. The diagnosis support program 76 stored in the storage medium is installed in the computer 54. The processor 62 executes the diagnosis support processing in accordance with the diagnosis support program 76.

In the embodiment described above, the computer 54 is described as an example, but the technology of the present disclosure is not limited to this, and a device including an ASIC, an FPGA, and/or a PLD may be applied instead of the computer 54. A combination of a hardware configuration and a software configuration may be used instead of the computer 54.

The following various processors can be used as a hardware resource for executing the diagnosis support processing in the embodiment described above. Examples of the processor include a processor as a general-purpose processor that executes software, that is, a program, to function as the hardware resource executing the diagnosis support processing. Examples of the processor also include a dedicated electronic circuit as a processor having a dedicated circuit configuration designed to execute specific processing, such as an FPGA, a PLD, or an ASIC. Any processor has a memory built in or connected to it, and any processor executes the diagnosis support processing by using the memory.

The hardware resource for executing the diagnosis support processing may be configured by one of the various processors or by a combination of two or more processors of the same type or different types (for example, a combination of a plurality of FPGAs or a combination of a processor and an FPGA). The hardware resource for executing the diagnosis support processing may also be one processor.

A first example of the configuration in which the hardware resource is configured by one processor is an aspect in which one processor is configured by a combination of one or more processors and software, and this processor functions as the hardware resource for executing the diagnosis support processing. As a second example, as typified by an SoC or the like, there is a form in which a processor that implements all functions of a system including a plurality of hardware resources executing the diagnosis support processing with one IC chip is used. As described above, the diagnosis support processing is implemented by using one or more of the various processors as the hardware resource.

Further, specifically, an electronic circuit obtained by combining circuit elements, such as semiconductor elements, can be used as the hardware structure of the various processors. Further, the diagnosis support processing is merely an example. Therefore, it goes without saying that unnecessary steps may be deleted, new steps may be added, or the processing order may be changed within a range that does not deviate from the gist.

The above-described contents and the above-shown contents are the detailed description of the parts according to the technology of the present disclosure, and are merely examples of the technology of the present disclosure. For example, the description of the configuration, the function, the operation, and the effect is the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate understanding of the parts according to the technology of the present disclosure, the description of common technical knowledge or the like, which does not particularly require the description for enabling the implementation of the technology of the present disclosure, is omitted in the above-described contents and the above-shown contents.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All of the documents, the patent applications, and the technical standards described in the present specification are incorporated into the present specification by reference to the same extent as in a case in which the individual documents, patent applications, and technical standards are specifically and individually stated to be described by reference.

What is claimed is:

1. A diagnosis support device comprising:
a processor, wherein:
the processor is configured to:
acquire an ultrasound image,
display the acquired ultrasound image on a display device, and
display, in the ultrasound image, a first mark capable of specifying a lesion region detected from the ultrasound image within the ultrasound image and a second mark capable of specifying an organ region detected from the ultrasound image within the ultrasound image, and
the first mark is displayed in a state of being emphasized more than the second mark.

2. The diagnosis support device according to claim 1, wherein:
the first mark is a mark capable of specifying an outer edge of a first range in which the lesion region is present.

3. The diagnosis support device according to claim 2, wherein:
the first range is defined by a first rectangular frame that surrounds the lesion region.

4. The diagnosis support device according to claim 3, wherein:
the first rectangular frame is a rectangular frame that circumscribes the lesion region.

5. The diagnosis support device according to claim 3, wherein:
the first mark is a mark in which at least a part of the first rectangular frame is formed in a visually specifiable manner.

6. The diagnosis support device according to claim 3, wherein:
the first rectangular frame surrounds the lesion region in a rectangular shape as seen in front view, and
the first mark is composed of a plurality of first images assigned to a plurality of corners including at least opposite corners of four corners of the first rectangular frame.

7. The diagnosis support device according to claim 1, wherein:
the second mark is a mark capable of specifying an outer edge of a second range in which the organ region is present.

8. The diagnosis support device according to claim 7, wherein:
the second range is defined by a second rectangular frame that surrounds the organ region.

9. The diagnosis support device according to claim 8, wherein:
the second rectangular frame is a rectangular frame that circumscribes the organ region.

10. The diagnosis support device according to claim 8, wherein:
the second mark is a mark in which at least a part of the second rectangular frame is formed in a visually specifiable manner.

11. The diagnosis support device according to claim 8, wherein:
the second rectangular frame surrounds the organ region in a rectangular shape as seen in front view, and
the second mark is composed of a plurality of second images assigned to center portions of a plurality of sides including at least opposite sides of four sides of the second rectangular frame.

12. The diagnosis support device according to claim 1, wherein:
the ultrasound image is a moving image including a plurality of frames, and
in a case in which N is a natural number equal to or larger than 2, the processor is configured to display the first mark in the ultrasound image in a case in which the lesion region is detected from N consecutive frames among the plurality of frames.

13. The diagnosis support device according to claim 1, wherein:
the ultrasound image is a moving image including a plurality of frames, and
in a case in which M is a natural number equal to or larger than 2, the processor is configured to display the second mark in the ultrasound image in a case in which the organ region is detected from M consecutive frames among the plurality of frames.

14. The diagnosis support device according to claim 1, wherein:
the ultrasound image is a moving image including a plurality of frames,
in a case in which N and M are natural numbers equal to or larger than 2, the processor is configured to:
display the first mark in the ultrasound image in a case in which the lesion region is detected from N consecutive frames among the plurality of frames, and
display the second mark in the ultrasound image in a case in which the organ region is detected from M consecutive frames among the plurality of frames, and
N is a value smaller than M.

15. The diagnosis support device according to claim 1, wherein:
the processor is configured to notify of detection of the lesion region by causing a sound reproduction device to output a sound and/or a vibration generator to generate a vibration in a case in which the lesion region is detected.

16. The diagnosis support device according to claim 1, wherein:
wherein the processor is configured to:
display a plurality of screens including a first screen and a second screen on the display device,
display the ultrasound image on the first screen and the second screen, and
separately display the first mark and the second mark in the ultrasound image on the first screen and in the ultrasound image on the second screen.

17. The diagnosis support device according to claim 1, wherein:
the processor is configured to detect the lesion region and the organ region from the ultrasound image.

18. An ultrasound endoscope comprising:
the diagnosis support device according to claim 1; and
an ultrasound endoscope body to which the diagnosis support device is connected.

19. A diagnosis support method comprising:
acquiring an ultrasound image;
displaying the acquired ultrasound image on a display device; and
displaying, in the ultrasound image, a first mark capable of specifying a lesion region detected from the ultrasound image within the ultrasound image and a second mark capable of specifying an organ region detected from the ultrasound image within the ultrasound image,
wherein the first mark is displayed in a state of being emphasized more than the second mark.

20. A non-transitory computer-readable storage medium storing a program for executable by a computer to execute a process comprising:
acquiring an ultrasound image;
displaying the acquired ultrasound image on a display device; and
displaying, in the ultrasound image, a first mark capable of specifying a lesion region detected from the ultrasound image within the ultrasound image and a second mark capable of specifying an organ region detected from the ultrasound image within the ultrasound image,
wherein the first mark is displayed in a state of being emphasized more than the second mark.
